(19) United States
(12) Patent Application Publication
(10) Pub. No.: US 2026/0176606 A1
(43) Pub. Date: Jun. 25, 2026

(54) PROTEIN WITH XYLANASE ACTIVITY

(71) Applicant: ADISSEO FRANCE S.A.S., Antony (FR)

(72) Inventors: Dominique Louis, Orsay (FR); Nina Sokoloff, Orsay (FR); Karine Bastard, Orsay (FR); Virginie Neugnot, Toulouse (FR)

(21) Appl. No.: 18/833,521

(22) PCT Filed: Feb. 3, 2023
(86) PCT No.: PCT/FR2023/050148

§ 371 Date: Jul. 26, 2024

(30) Foreign Application Priority Data
Feb. 4, 2022 (FR) 22/00999

(51) Int. Cl. C12N 9/24; A23K 20/189

(52) U.S. Cl. CPC C12N 9/2482; A23K 20/189; C12Y 302/01008

(57) ABSTRACT

The invention concerns a thermostable protein expressing at least one xylanase activity, characterized in that it comprises or consists of a peptide sequence represented by SEQ ID NO: 1 including at least the L34C and R38C mutations, a polynucleotide encoding for such a protein, a vector allowing the expression of this polynucleotide in a host cell, a so-called host cell, as well as any application in particular in animal nutrition of said protein.

The present invention concerns a protein having at least one xylanase activity.

By xylanase activity, according to the invention is meant a protein or an enzyme, of natural or modified origin, capable of promoting the degradation, at least partially, of xylan, by hydrolysis, by releasing xylose or an intermediate molecule capable of being transformed into xylose by another enzyme, under specific conditions. It is known several enzymes, or xylanases, meeting this definition, which differ by their specific mode of action of certain chemical bonds, including endoxylanase or endo-1,4-β-xylanase and beta-xylosidase.

Xylan is a so-called non-starch polysaccharide (NSP) abundant in nature, it is a majority constituent of hemicellulose. It is widely present in human and animal foods due to diets that may be rich in plant fibers. However, neither man nor mammals are able to produce xylanase or at least not in sufficient quantity for efficient hydrolysis, and a supply of xylanase in the diet may be desirable to promote the digestion of carbohydrates. Thus, xylanase supplementation is widely used in animal nutrition, particularly for livestock, in order to optimize the use of the fibrous part of the feed by the animal, leading to an increase in growth performance. We are talking about a zootechnical additive.

The action of a xylanase being specific, a mixture of xylanases with preferably complementary mechanisms is advantageously used. A xylanase or a mixture of xylanases is (are) also generally used in combination with one or more other enzymes in order to degrade other polysaccharides present in the feed, such as a beta-glucanase, an amylase, a protease or in order to confer an additional benefit to the additive, such as a phytase.

The xylanases can be produced naturally by many organisms such as plants, algae, gastropods, arthropods, yeasts, fungi, bacteria, protozoa; However, they have been the subject of numerous studies to improve their properties and the industry mainly uses proteins produced by genetically modified organisms.

If, in the present text, the interest of a xylanase according to the invention is illustrated in an application to animal nutrition, the invention is not limited there and the purpose of such a xylanase can be quite different by example in the fields of waste treatment, the pulp and paper, textiles, the production of biofuels industry, the agri-food industry like in bread-making.

As a zootechnical additive, the xylanase or the mixture of enzymes containing it must resist the particularly thermal conditions of the environment in which it is used, namely those of the digestive system of the animal whose temperature can vary from around 35° C. to 42° C., or even more. Moreover, the xylanase or the mixture of enzymes containing it is mixed with the feed and therefore subjected to a transformation method for its shaping, including steps of mechanical treatment and heat treatment. The use of heat to shape the feed can reach temperatures of around 80° C. or more and it is essential that the enzyme(s) are not denatured by such treatment to preserve their hydrolytic potential and therefore their benefit (J. Inborr et al. Animal Feed Science and Technology: Stability of feed enzymes to steam pelleting during feed processing, 46 (1994) pp 179-196). If xylanase of natural origin is not affected by temperatures up to 50° C., this is no longer the case at higher temperatures, and its activity can drop drastically. Indeed, the exposure to such temperatures causes irreversible modifications of the enzyme, in particular by destruction of intramolecular bonds in the tertiary or quaternary protein structures, inhibiting the activity of the enzyme.

We are therefore looking for enzymes with xylanase activity, thermostable, capable of withstanding the heat treatments applied to it and which have a xylanase activity greater than that of the natural enzyme exposed to the same conditions.

The development of a thermostable xylanase that can be used on an industrial scale as well as in the field of animal nutrition nevertheless faces another obstacle, that of profitable production.

The document Zhe Xu et al., Recent advances in the improvement of enzyme thermostability by structure modification, 2020, vol. 40, no. 1 pp 83-98, reports the latest developments on the development of thermostable enzymes in connection with their secondary and tertiary structure. Mutations are made to generate covalent and non-covalent interactions likely to give the enzyme greater thermostability. If a relationship does indeed exist between molecular structure and thermostability, improving the thermostability of an enzyme remains a difficulty despite the tools of protein engineering.

The invention provides a mutated xylanase which is a good compromise between thermostability and industrial production. Indeed, after an exposure to temperatures of around 80° C., the enzyme of the invention retains strong xylanase activity, under the environmental conditions to which it is subjected in the digestive system of an animal, such as an acidic pH, a temperature of approximately 35° C. to 42° C., while being able to be produced in yields in the range of those of the natural enzyme.

The starting point of the invention is a recombinant xylanase EvAuXyn11A having greater thermostability than the xylanases of family 11 of glycoside hydrolases (GH) naturally produced and which is described in document CN102492676A. EvAuXyn11A is obtained from the xylanase AuXyn11 whose N-terminal sequence has been substituted by that of EvXynATS.

According to the invention, starting from a thermostable xylanase XYN3 identified by SEQ ID NO: 1, one or more specific mutations were introduced, resulting in an unexpected increase in the thermostability of the xylanase.

Specifically, the invention concerns a thermostable protein expressing at least one xylanase activity, said protein comprising or consisting of a peptide sequence represented by SEQ ID NO: 1 comprising at least the L34C and R38C mutations.

As shown by the results presented later in the text, these mutations lead, after exposure for 50 minutes to 80° C., to more than a doubling of the xylanase activity of the XYN3 protein measured at 50° C.

It was further discovered that complementary mutations could contribute to an increase in the production yield of the protein and/or an improvement in its thermostability. Thus, the invention concerns a thermostable protein expressing at least one xylanase activity, said protein comprising or consisting of a peptide sequence represented by SEQ ID NO: 1 which includes at least the L34C and R38C mutations and one or more mutations selected from T112E, Q145E, S187R. The presence of one of these complementary mutations, or even two or even three, leads to a highly thermostable protein without significantly affecting its production yield.

The invention also relates to a polynucleotide encoding for a protein of the invention as defined above.

By polynucleotide encoding for a protein, it is understood a sequence of nucleic acid(s) of DNA, cDNA, or synthetic DNA which responds to the open reading frame (ORF) which is translated into said protein, in particular, it can comprise a start codon and a stop codon. It may also include any additional nucleic sequence necessary for the expression of said protein; this will notably involve regulatory sequences including at least one or more promoters and signals for starting and stopping transcription or translation. It is of course up to the general knowledge of those skilled in the art to use all the tools, methods and techniques to which they have access in this field of biology.

Thus, a polynucleotide of the invention comprises or consists of a nucleotide sequence SEQ ID No: 2 having at each of positions 100-102 and 112-114, the codon TGT or TGC encoding for cysteine, to take into account the degeneration of the genetic code. Another polynucleotide of the invention comprises or consists of SEQ ID NO: 2 having at each of positions 100-102 and 112-114, the codon TGT or TGC encoding for cysteine, and furthermore having at least one of the following mutations, or any two of the mutations, or even all three of the following mutations:
at the position 334-336, the GAA or GAG codon encoding for glutamic acid,
at the position 433-435, the GAA or GAG codon encoding for glutamic acid,
at the position 559-561, the codon CGT, CGC, CGA, CGG, AGA, AGG encoding for arginine.

It is of course within the general knowledge of those skilled in the art to deduce from a protein of the invention the nucleotide sequence of the corresponding reading frame.

Yet another object of the invention is an expression vector such as a plasmid, comprising a polynucleotide of the invention, and at least the elements necessary for its expression in a host cell.

The invention also concerns a host cell comprising an aforementioned expression vector.

As previously said, a protein of the invention finds application, without being limited to it, in food and in particular in animal nutrition. In this regard, the invention relates to a zootechnical additive comprising at least one such protein, and also to a feed for animal nutrition comprising at least one such zootechnical additive. A preferred target is monogastric livestock.

Thus, any use of a protein of the invention for feeding animals, for example livestock and in particular monogastric livestock, or for preparing a zootechnical additive as defined above or a feed as defined above is part of the invention.

The invention also concerns a method for manufacturing a protein of the invention, comprising at least one step of culturing a host cell defined above under conditions suitable for the production of said protein and a step of recovery of said protein.

The invention is illustrated in the examples which follow in support of the figures according to which:

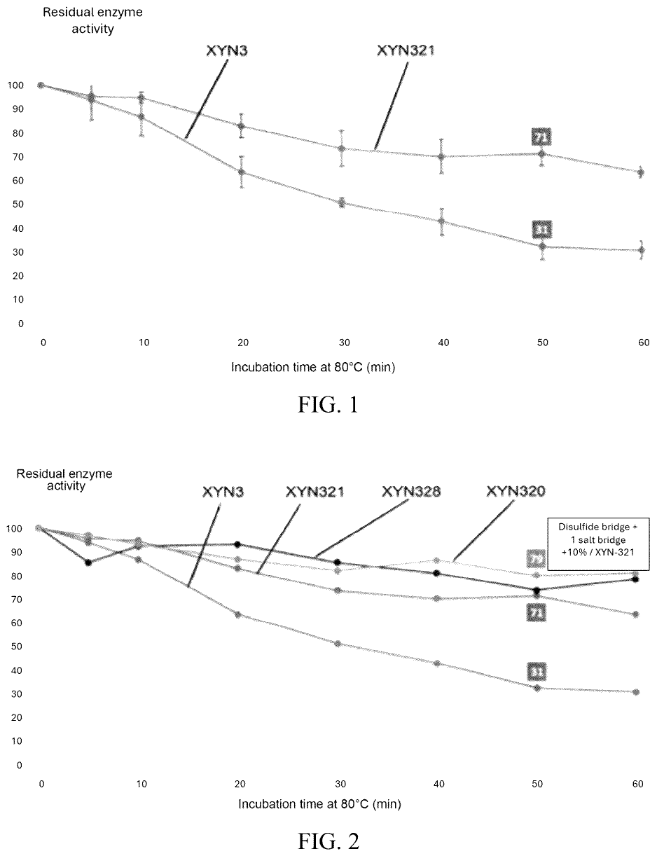

FIG. 1 represents the thermostability of the XYN321 variant compared to the reference XYN3 protein;

FIG. 2 represents the thermostability of the XYN321, XYN328 and XYN320 variants compared to the reference XYN3 protein;

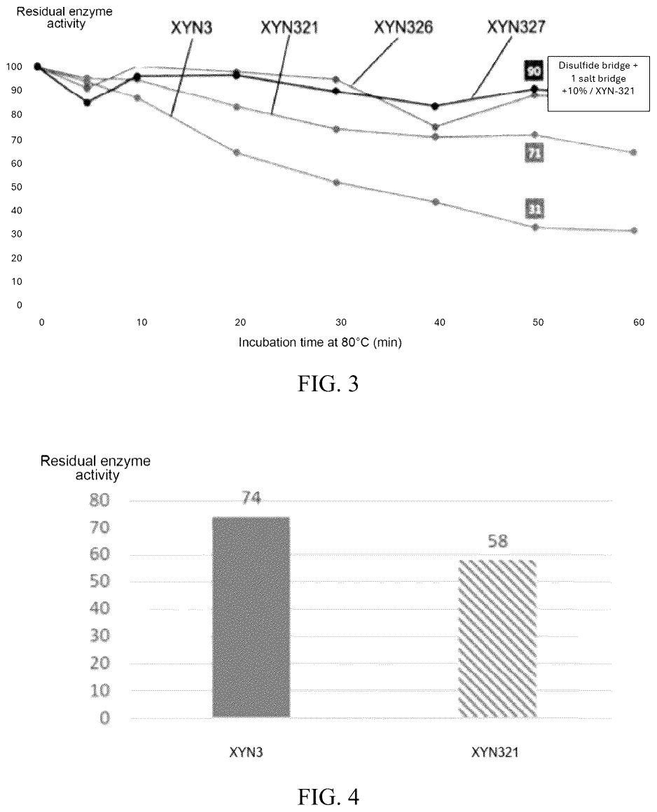

FIG. 3 represents the thermostability of the XYN321, XYN326 and XYN327 variant compared to the reference XYN3 protein;

FIG. 4 illustrates the thermostability of the XYN321 variant compared to the reference XYN3 protein during the manufacture of a feed according to the invention.

### EXAMPLE 1: MANUFACTURING OF XYN3 VARIANTS

The variants were obtained by conventional recombinant DNA technology implementing the steps of providing a nucleic acid sequence encoding for the protein of interest, inserting said sequence into an expression vector and introducing said vector in an appropriate cellular system allowing, by culture, to produce the recombinant protein.

The genes encoding the variants were obtained by chemical synthesis of the nucleotide sequences.

These technologies are entirely part of the general knowledge of those skilled in the art. For all purposes, reference may be made to AL Demain et al., Biotechnology Advances, Production of Recombinant Proteins by Microbes and Higher Organisms, 27 2009, pp 297-306 for the production of proteins, and to RA Hugues et al., Methods in Enzymology, Volume 498, 2011, Elsevier Inc. Gene Synthesis: Methods and Applications, Chapter 12, pp 277-309 for the synthesis of 10 genes involved in the production of variants.

The variants listed in Table 1 below were manufactured.

TABLE 1

| | Mutations on SEQ ID NO: 1 | | | | | | | |
|---|---|---|---|---|---|---|---|---|
| Protein | Q3E/L34R | D24C/G47C | L34C/R38C | T112E | Q145E | T154R | S187R | T179C/S188C |
| XYN3 | — | — | — | — | — | — | — | — |
| (reference) | | | | | | | | |
| XYN321 | — | — | x | — | — | — | — | — |
| XYN320 | — | — | x | — | — | — | x | — |
| XYN328 | —— | — | x | x | — | — | — | — |
| XYN326 | | — | x | x | x | — | — | — |
| XYN327 | — | — | x | x | — | — | x | — |
| XYN383 | — | — | — | x | — | — | — | — |
| (outside inv.) | | | | | | | | |
| XYN384 | — | — | — | — | x | — | — | — |
| (outside inv.) | | | | | | | | |
| XYN386 | — | — | — | — | — | — | x | — |
| (outside inv.) | | | | | | | | |
| XYN376 | — | — | — | x | x | — | — | — |
| (outside inv.) | | | | | | | | |
| XYN380 | — | — | — | — | x | — | x | — |
| (outside inv.) | | | | | | | | |
| XYN374 | — | — | — | x | x | — | x | — |
| (outside inv.) | | | | | | | | |
| XYN388 | — | — | — | x | x | — | x | |
| (outside inv.) | | | | | | | | |
| XYN32 | — | — | — | — | — | — | — | x |
| (outside inv.) | | | | | | | | |
| XYN391 | x | — | — | x | x | x | x | x |
| (outside inv.) | | | | | | | | |
| XYN324 | — | x | — | — | — | — | — | — |
| (outside inv.) | | | | | | | | |

### EXAMPLE 2: INFLUENCE OF THE MUTATION(S) ON THE PRODUCTION OF THE PROTEIN, ON ITS Xylanase Activity And On Its Thermostability

### 2.1-Influence on the Production

The production of each of the variants listed in Table 1 is evaluated on the basis of the enzymatic activity assayed according to the DNS method. This method consists of incubating a solution containing the variant in the presence of beech xylan for 10 minutes at 50° C. Xylanase cuts the xylan chain (composed of xyloses) into shorter chains or oligosaccharides and thus releases reducing ends or reducing xyloses. These are determined by dinitrosalicylic acid (DNS) at 95° C. and at alkaline pH, the DNS is attached to the reducing oses revealing a yellow-orange color proportional to the quantity of reduced oses and measurable with the spectrophotometer at 540 nm. Each measurement is carried out in duplicate. Quantification is carried out using a standard range of xylose, a reducing sugar.

### 2.2-Influence on Xylanase Activity after Incubation at 80° C.

The thermal stability was studied in vitro by incubating the supernatants of cultures containing a sufficient quantity of each enzyme at 80° C. and by measuring their activity, after an incubation of 0, 5, 10, 20, 30, 40, 50 and 60 minutes at 80° C. Immediately after the incubation at 80° C., the samples are placed on ice (+4° C.) then centrifuged to bring down all the supernatant to the bottom of the tube. The supernatant is then diluted in 25 mM sodium acetate buffer pH 4.0+0.1% BSA so as to obtain a concentration of xylanase suitable for the DNS assay. Xylanase activity is then measured by DNS for each incubation time.

The results of xylanase production and activity at 80° C. are presented in [Table 2] below.

TABLE 2

| | Production | % Initial activity after |
|---|---|---|
| Protein | U · mL⁻¹ | 50 min at 80° C. |
| XYN3 (reference) | 18 | 30 |
| XYN321 | 13 | 69 |
| XYN320 | 9 | 80 |
| XYN328 | 14 | 73 |
| XYN326 | 7 | 86 |
| XYN327 | 13 | 90 |
| XYN383 (outside invention) | 7 | 5 |
| XYN384 (outside invention) | 10 | 5 |
| XYN386 (outside invention) | 12 | 6 |
| XYN376 (outside invention) | 16 | 19 |
| XYN380 (outside invention) | 11 | 18 |
| XYN374 (outside invention) | 11 | 50 |
| XYN388 (outside invention) | 13 | 46 |
| XYN32 | 43 | 17 |
| XYN391 | 16 | 26 |
| XYN324 | — | 0 |

Based on [Table 2] and the figures, it is observed the following phenomena:
[FIG. 1]: the L34C and R38C (XYN321) mutations generating a disulfide bridge make it possible to considerably increase the stability of the xylanase: the residual activity after 50 minutes of incubation at 80° C. is 71% on average compared to 31% for native protein. Even if they lead to a slight reduction in the production of the XYN321 enzyme of the XYN-3 enzyme expressed under the control of the same promoters, this is insignificant compared to the thermostability conferred on the protein;
[FIG. 2]: the combination of the L34C and R38C (XYN321) mutations generating a disulfide bridge with a T112E or S187R mutation generating a salt bridge leads to an increase in the stability of the protein by 10%;
[FIG. 3]: the combination of the L34C and R38C (XYN321) mutations generating a disulfide bridge with the T112E mutation generating a first salt bridge and one or other of the Q145E and S187R mutations generating a second salt bridge leads to an increase protein stability by 25%.

It further appears from [Table 2] that other mutations causing the creation of disulfide bridges have the effect of lowering the thermostability of the enzyme compared to the reference XYN3 enzyme (variants XYN32, XYN391) and that certain lead to the inactivation of the enzyme (XYN324 variant). It is also noted that the preferential mutations according to the invention (T112E, Q145E and S187R) in the absence of the L34C and R38C mutations but in the presence of the T179C/S188C mutations generating disulfide bridges, only modestly compensate for the thermostability of the enzyme.

### EXAMPLE 3: INFLUENCE OF THE MUTATION(S) OF A XYLANASE ACCORDING TO THE INVENTION ON ITS ENZYMATIC ACTIVITY MEASURED ON A FEED SAMPLE (IN VIVO TEST)

### Preparation of a Feed According to the Invention

A suitable method is described in document WO2009/019335A1.

### Manufacturing of Enzyme Particles

The enzyme in liquid form is deposited on a support consisting of flour (or any other suitable support) by spray-drying, involving spraying the enzyme onto the flour and co-drying, for example by atomization (or spray-drying) to obtain a powder. This is then wet granulated by impregnation with a coating agent, then covered with a protective coating agent and then dried.

### Feed Pelleting

The enzyme particles and a nutritional base such as a mixture of wheat, soybean meal, extruded soybeans, palm oil, calcium carbonate, dicalcium phosphate, salt and a premix containing methionine, are mixed and heated to a temperature of 60 to 100° C., preferably selected between 7° and 90° C. The mixture is then pelletized in a pellet press, the pellets obtained are cut to the desired length then dried, to obtain the granulated feed.

Measurement of the xylanase activity of the feed thus manufactured

After extraction of the enzyme contained in the granulated feed, the residual xylanase activity is measured using the viscosimetry method. This method consists of incubating a solution containing the variant in the presence of wheat arabinoxylan at 30° C. and determining the viscosity of the mixture using a microviscosimeter for 20 minutes. The xylanase activity corresponds to the degradation of arabinoxylan to oligoarabinoxylan and is proportional to the reduction in substrate viscosity in the presence of the enzyme. Each measurement is carried out in duplicate.

The results are shown in [FIG. 4]; they demonstrate the stability of the xylanase activity of the XYN321 protein compared to the reference XYN3 protein.

1. A thermostable protein expressing at least one xylanase activity, characterized in that it comprises or consists of a peptide sequence represented by SEQ ID NO: 1 comprising at least the mutations L34C and R38C.

2. The protein according to claim 1, characterized in that the peptide sequence SEQ ID NO: 1 includes at least one mutation selected from T112E, Q145E and S187R.

3. The protein according to claim 2, characterized in that the peptide sequence SEQ ID NO: 1 includes at least the T112E mutation.

4. The protein according to claim 3, characterized in that the peptide sequence SEQ ID NO: 1 includes at least the S187R mutation.

5. A polynucleotide encoding a protein according to any one of claims 1 to 4.

6. The polynucleotide according to claim 5, characterized in that it comprises the nucleotide sequence SEQ ID NO: 2 presenting at each of positions 100-102 and 112-114, the codon TGT or TGC encoding for cysteine.

7. The polynucleotide according to claim 6, characterized in that the nucleotide sequence SEQ ID NO: 2 also has at least one of the following mutations, or any two of the mutations, or even the three following mutations:
at the position 334-336, the GAA or GAG codon encoding for glutamic acid,
at the position 433-435, the GAA or GAG codon encoding for glutamic acid,
at the position 559-561, the codon CGT, CGC, CGA, CGG, AGA, AGG encoding for arginine.

8. An expression vector such as a plasmid comprising a polynucleotide according to any one of claims 5 to 7, and at least the elements necessary for its expression in a host cell.

9. A host cell comprising an expression vector according to claim 8.

10. A zootechnical additive comprising at least one protein according to any one of claims 1 to 4.

11. A feed for animal nutrition comprising at least one zootechnical additive according to claim 10.

12. (canceled)

13. A method for producing a protein according to any one of claims 1 to 4, comprising at least one step of culturing a host cell according to claim 9 under conditions suitable for the production of said protein and a step of recovering said protein.


SEQUENCE LISTING (2 sequences)

SEQ ID NO: 1 - protein, 193 aa, synthetic construct
  REGION 1..193: note = Recombinant protein
NAQTCLTSPQTGFHNGFFYSFWKDSPGTVNFCLLEGGRYTVEWSNVGNFVGGKGWNPGSAQDITYSGTFTPSGNGYLSVYGWTTDPLIEYYIVESYGDYNPGSGGTYKGTVTSDGSVYDIYTATRTNAASIQGTATFTQYWSVRQNKRVGGTVTTSNHFNAWAKLGMNLGTHNYQIVATEGYQSSGSSSITVQ

SEQ ID NO: 2 - DNA, 582 bp, synthetic construct
  misc_feature 1..582: note = artificial sequence
  source 1..582: mol_type = other DNA
aatgcccaaacatgtttgacatctccacaaacagggtttcacaatggttttttctattcattttggaaagattcacccggtactgtcaatttttgcttactagaaggaggtagatacacagtagagtggagtaatgtgggcaactttgtcggaggtaaaggctggaatccaggtagcgctcaggatattacttattccggcaccttcactccttcaggtaatggttacttgtccgtctacggctggacgactgaccctcttattgaatactatattgtggaaagctatggcgactataacccggggtctggaggaacgtataaggggacggtaacttcggatggttctgtttatgatatatatacagcgactaggaccaatgctgcttcgatacagggaaccgccactttcacacagtactggtcagttcgtcaaaataagagagttggaggtactgttactacatctaaccatttcaacgcatgggcaaaattaggtatgaacctgggtacacataactaccaaatcgttgcaaccgaaggttaccaatccagtggtagttcttctatcaccgtacaataa